(19) United States
(12) Patent Application Publication
(10) Pub. No.: US 2026/0063543 A1
(43) Pub. Date: Mar. 5, 2026

(54) SIZE-BASED HIERARCHICAL EXTRACTION AND IDENTIFICATION METHOD FOR MICROPLASTICS IN BIVALVES FROM DEEP-SEA METHANE SEEPS

(71) Applicant: SOUTHERN MARINE SCIENCE AND ENGINEERING GUANGDONG LABORATORY (GUANGZHOU), Guangzhou (CN)

(72) Inventors: Jingchun FENG, Guangzhou (CN); Canrong LI, Guangzhou (CN); Si ZHANG, Guangzhou (CN); Zhifeng YANG, Guangzhou (CN)

(73) Assignee: SOUTHERN MARINE SCIENCE AND ENGINEERING GUANGDONG LABORATORY (GUANGZHOU), Guangzhou (CN)

(21) Appl. No.: 19/312,378

(22) Filed: Aug. 28, 2025

(30) Foreign Application Priority Data
Aug. 28, 2024 (CN) 202411192030.7

(51) Int. Cl. G01N 21/3563; C12Q 1/37; G01N 1/40; G01N 21/35; G01N 21/65; G01N 33/44; G01N 33/483

(52) U.S. Cl. CPC G01N 21/3563; C12Q 1/37; G01N 1/4044; G01N 21/65; G01N 33/442; G01N 33/4833; G01N 2001/4088; G01N 2021/3572; G01N 2021/3595; G01N 2333/976

(57) ABSTRACT

A size-based hierarchical extraction and identification method for microplastics in bivalves from deep-sea methane seeps is provided. The method freeze-dries and dehydrates biological tissues, uses a pH phased enhancement enzyme-hydrogen peroxide mixed digestion solution, hierarchical progressive vacuum filtration, size-based advantage identification, and other experimental steps to extract microplastics contained in bivalves in extreme environments non-destructively and in a classified manner, with the objective of achieving quantitative and qualitative analysis of the full-scale range of microplastics in bivalves.

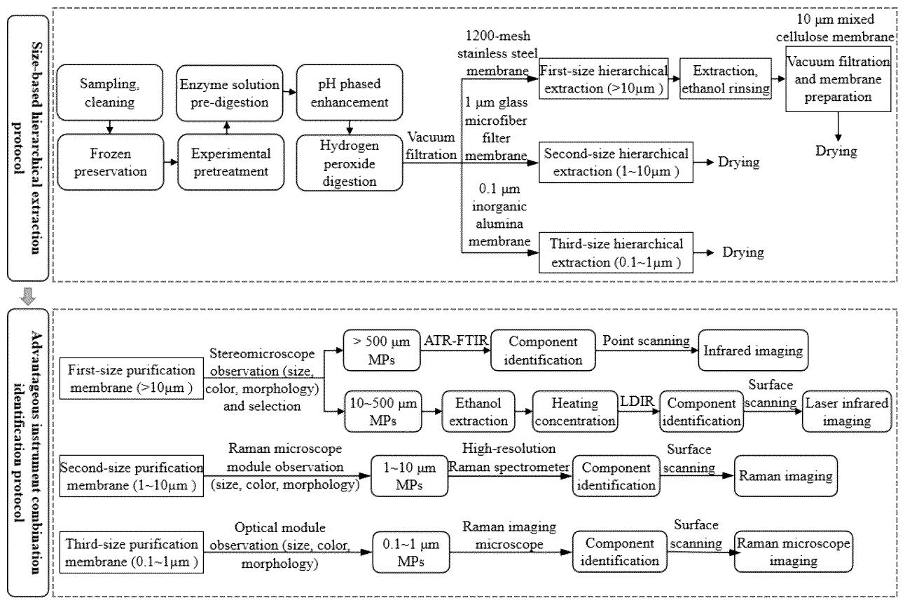

### CROSS REFERENCE TO THE RELATED APPLICATIONS

This application is based upon and claims priority to Chinese Patent Application No. 202411192030.7, filed on Aug. 28, 2024, the entire contents of which are incorporated herein by reference.

### TECHNICAL FIELD

The present application relates to the technical fields of biological anatomy and extraction and detection of new pollutants in deep-sea extreme environments, and specifically relates to a size-based hierarchical extraction and identification method for microplastics in bivalves from deep-sea methane seeps.

### BACKGROUND

Deep-sea extreme environments refer to regions in the ocean depths, typically at water depths exceeding 1000 meters. These regions differ from conventional aquatic environments, characterized by high pressure, low temperature, near darkness, scarce nutrients, and extreme chemical conditions (such as methane or hydrogen sulfide concentrations). This unique environment nurtures various special seafloor habitats including deep-sea plains, seamounts, hydrothermal vents, cold seeps, and abyssal zones. However, these special extreme habitats all possess different accumulation mechanisms for trapping pollutants. They either concentrate pollutants contained in bottom waters through hydrodynamic forces provided by thermohaline circulation, or increase the loading gravity of pollutants through biological etching effects, or condense pollutants to form binding barriers through chemical mineralization reactions, or concentrate pollutants due to local phase pressure differences caused by powerful upwelling currents. All these constitute the complex control patterns of ocean currents and pollutant occurrence distribution in deep-sea extreme environments. The average depth of the global ocean exceeds 3600 m, with over 90% of sea areas located in deep-sea extreme environments. Continental shelf coastal currents and subducting water flows from river estuaries serve as major inlets for surface ocean currents, transporting land-based pollutants to ocean margins. When surface ocean currents sink and converge in continental slope areas, gravity flows become unsealed through topography and join the ranks of seafloor turbidity currents, transporting pollutants from surface and middle-layer seawater to the deep seafloor. Meanwhile, sea surface wind friction and geostrophic deflection force drive surface ocean currents toward the deep sea, and Langmuir turbulence induced by sea surface waves controls the rate of vertical transport of surface pollutants to the water column. Conversely, as near-bed shear stress increases in seafloor surface sediments, pollutants in sediment surfaces are reactivated and transported as circulating bed loads, with pollutants resuspending in bottom seawater and participating in the water mass circulation of ocean currents. Ancient water masses in the ocean depths serve as bridges connecting material transport between seafloor extreme habitats and surface-middle layer ocean currents, with accumulated pollutant particle concentrations, such as plastic microparticles, being activated and recycled within this system for decades unchanged. Therefore, deep-sea extreme environments more easily form long-term accumulation sites for plastic microparticles.

Microplastics are defined as plastic microparticles of 0.0001-5 mm, serving as the main carrier of marine plastic pollution. More than 10 million tons of plastic products enter global oceans annually, with plastics present in seawater being fragmented into small-diameter microplastics through light radiation, mechanical stress wear, biological fouling, and physical erosion. However, plastic particles accumulated on the sea surface only account for about 1% of the predicted marine plastic inventory, with the remaining 13.5% entering deep-sea extreme environments in the form of microplastics. Most microplastics entering extreme environments exhibit sparse porous surface structures, with sizes≤50 μm accounting for over 50% of total seawater microplastic abundance. A small number of large-size (≥1 mm) microplastics that traverse ocean current impacts but rarely fracture are almost entirely composed of extremely flexible polyamide materials. In the vast deep seafloor, smaller-size (<250 μm) microplastics are more easily captured by extreme environments. Bottom water microplastics stored in extreme environments gradually form clearly distinct size-grade mechanisms through long-term temporal enrichment. The first size grade is generally <50 μm, followed by 50-100 μm, 100-250 μm, 250-500 μm, 500-1000 μm, and 1000-5000 μm, with the first three size grades encompassing nearly 80% of total microplastic size volume. However, current instrumental methods for identifying microplastic types all have their advantageous identification ranges and cannot completely detect collected suspected microplastics using a single device. To quantify small-size microplastics in deep-sea extreme environments, completely extracting suspected microplastics by size grades has become an alternative innovative concept for comprehensive surveying of full-size range microplastic extraction.

Deep-sea methane seeps are a special case existing in deep-sea extreme environments, typically appearing in continental margins, seafloor fracture zones, and hydrate burial areas. The formation of methane seeps depends on sudden changes in seafloor fluid environments or geological structural activities that cause generated gaseous methane to accumulate in sediment pores and leak upward through methane channels. Meanwhile, the sulfate-methane transition zone (SMTZ) composed of methane anaerobic oxidation bacteria and sulfate-reducing bacteria in sediment rock layers effectively weakens upwelling methane gas flows, accompanied by large amounts of metal elements and hydrogen sulfide gas escape, forming specific cold seep fluid gushing seep areas on the seafloor. Unlike hydrothermal vents, cold seep fluid temperatures are slightly lower than surrounding seawater temperatures. Nutrients transmitted from rock layer primary producers provide carbon sources and elemental supply for chemosynthesis in methane seeps. Continuously seeping cold seep fluids nourish a major class of indicator organisms in methane seeps—bivalves, gradually forming special chemosynthetic ecosystems using methane and sulfides as special carbon sources and redox energy supply. Methane seeps are also hotspots for accumulating seawater microplastics. Besides large amounts of gushing cold seep plumes forming vacuum gas layers that gather surrounding seawater microplastics and methane bubble buoyancy effects migrating seafloor microplastics to shallow layers, bivalves inhabiting methane seeps also adsorb microplastics in bottom waters through filter-feeding and mucus capture mechanisms, transferring microplastics into organisms' digestive systems. Therefore, when assessing risks of deep-sea extreme ecosystem contamination by microplastics, bivalves are commonly used as model research organisms for measuring microplastic accumulation levels.

In methane seeps with continuous violent methane plume gushing, bivalves as seafloor organisms engulf bottom water flows through cilia on their gills, continuously adsorbing suspended substances (including microplastics) in water flows onto gill filter nets. Cold seep ecosystems with multiple microplastic enrichment capabilities serve as potential plastic sinks for both seawater and sediments, playing roles in reincorporating carbon from microplastics into marine carbon pools for storage. Meanwhile, bivalve gills and mantles secrete mucus due to life activities to irregularly capture particulate organic matter in filter nets. Most particulate microplastics are retained in gills, while small portions of captured microplastics enter stomach tissues along with organic matter for preliminary biofouling. Since microplastic particles are difficult to digest and absorb, most microplastics in stomach tissues transfer to intestines and are gradually worn into smaller-size microplastics through peristaltic motion. Plastics fragmented to nanoscale levels are absorbed through digestive system processes along with organic matter into blood, then migrate to other tissues and organs where they reaggregate into microplastics through tissue protein mediation. Microplastics accumulated in intestines eventually form partial pseudofeces for excretion or continue adhering within folded intestinal cells. Tracing migration routes of microplastics within bivalves, most studies show that ingested microplastic particles mainly accumulate in gills, digestive systems (stomach and intestines), mantles, and hepatopancreas, with gills and digestive systems being key sites for primary capture and transport of microplastics, while hepatopancreas and mantles serve as microplastic dissolution and deposition sites. Accumulation abundance of microplastics in various bivalve tissues is affected by size, with hepatopancreas/intestines considered final fragmentation sites for microplastics in bivalve tissues. Accumulation amounts of microplastics in these sites can rapidly increase in short periods. Besides unconscious transport by digestive systems, small-size microplastic particles also self-fragment, forming multiple overlays of local abundance. The distribution patterns of microplastic content in various tissues are approximately: hepatopancreas/intestines>gills>mantle>gonads>lips>foot>adductor muscles. However, not all bivalve species in methane seeps possess similar microplastic adsorption capabilities. Deep-sea mussels that can mobilize more gill ciliary cells for coordinated movement possess characteristics for adsorbing more seafloor water flow microplastics due to excessive filter-feeding efficiency. Vesicomyids that can secrete more viscous mucus amounts for capturing suspended particulate matter possess properties for more effectively capturing microplastic particles due to mucus secretion amounts. White clams that can use strong methane seep vents as advantageous ecological niches possess special capabilities for gathering surrounding aquatic environment microplastics due to gas-phase vacuums formed by cold seep fluids. Microplastics originally present in deep-sea extreme environments are mainly small-sized, but after intense fluid disturbance in methane seeps and filter-feeding adsorption by bivalves, microplastic size ranges within bivalves are further reduced, with most being <250 μm. Extraction schemes for small and medium-size microplastics in biological tissues urgently require higher detection requirements. Current schemes for extracting microplastics from bivalve biological tissues usually only refer to bivalves from ordinary aquatic environments or shallow-sea aquaculture, not involving discussions of bivalves in deep-sea extreme environments. However, bivalves nurtured in extreme environments have evolved highly specialized tissue organs, such as feet evolved with capabilities to sense methane fluids and withstand impacts; visceral masses developing strengthened muscle support structures to cope with high-pressure seafloor environments, causing stomach and digestive glands within tissues to twist together indistinguishably; enhanced extracellular matrix density between tissues to maintain cellular morphology under high pressure. This causes conventional bivalve digestion schemes to fail to achieve normal digestion states when applied to bivalves in extreme environments, resulting in low microplastic extraction efficiency and bringing incorrect cognition and understanding to properly recognizing microplastic abundance accumulated in methane seep bivalve tissues.

Through reviewing extensive literature on extracting microplastics from bivalves bred in ordinary aquatic environments, scholars commonly use alkaline digestion methods to digest dissected bivalve tissues and filter them. Microplastic extraction and identification steps are as follows:
\textcircled{1} Sample preservation: Clean captured bivalves with ultrapure water to remove salts, place cleaned bivalves in washed insulated glass jars, seal and preserve with ethanol solution, and promptly return to laboratory for processing;
\textcircled{2} Sample pretreatment: Repeatedly rinse bivalve surfaces multiple times with ultrapure water to wash away adsorbed impurities and sediment. Separate visceral mass, gills, and muscle bundles on dissection table, clean all separated soft tissues with ultrapure water, set aside for use;
\textcircled{3} Homogenate freeze-drying: Combine and mix dissected tissues from different parts, crush uniformly using homogenizer to obtain tissue homogenate, freeze-dry homogenate to obtain freeze-dried powder samples;
\textcircled{4} Constant temperature digestion: Weigh fixed mass of freeze-dried powder samples, add digestive solution (such as potassium hydroxide solution, sodium hydroxide solution, protease solution, hydrogen peroxide solution, nitric acid-perchloric acid solution, etc.), set digestion temperature at 40-60° C. with constant temperature shaking for 24 h;
\textcircled{5} Vacuum filtration: Filter digestion solution with stainless steel mesh/nitrocellulose filter membrane, repeat filtration multiple times, continuously wash adsorbed particles on mesh/filter membrane with ultrapure water or organic reagents (such as carbon tetrachloride, toluene, etc.);
\textcircled{6} Organic extraction: Place mesh/filter membrane in organic solvent for extraction, perform ultrasonic oscillation to elute attached microplastics, rinse with corresponding solvents to collect uniformly dispersed microplastic suspensions;
\textcircled{7} Vacuum suction filtration: Vacuum filter concentrated microplastic suspensions onto new nitrocellulose filter membranes, place in oven to dry for 3 h, obtain filter membrane samples enriched with microplastics;
\textcircled{8} Instrument identification: Place filter membranes under magnifying glass/microscope, visually select suspected microplastics and transfer to new membranes, record morphological information such as color, size, shape of microplastics. Then load suspected microplastics in the FTIR sample chamber and activate total reflection mode to identify their types and functional group information.

Currently, detection methods for analyzing microplastics in bivalves bred in general aquatic environments mostly follow the above procedures, with the following disadvantages: \textcircled{1} Using ultrapure water or organic solutions to clean or seal and preserve bivalves and their biological tissues. Bivalves surviving in deep-sea extreme environments have adapted to high-pressure, high-salt ecological conditions. Using ultrapure water to clean biological samples may cause strong changes in intracellular osmotic pressure, with continuously precipitated salts simultaneously promoting particle swelling effects of microplastics in cellular gaps. The extracellular matrix is cleared by large amounts of water, tissue structure is damaged, and microplastics may escape prematurely to external environments through damaged tissues, causing scientific illusions of low abundance. Using organic solvents for cleaning and preservation causes even more interference to the structure and morphology of microplastics in tissues. Some organic solvents (such as acetone) not only dissolve fluorescent additives on microplastic surfaces but also cause severe dehydration and protein denaturation in biological tissue cells. Microplastics residing within are subjected to strong external compression forces and more easily fracture to form smaller-size microplastics or nanoplastics, causing macroscopic phenomena of inflated abundance; \textcircled{2} Other patents commonly obtain tissue homogenate through grinding/mechanical crushing to improve organic matter digestion efficiency. The biggest drawback of this method is destroying fibrous microplastics adsorbed in biological tissues, violently wearing fibrous microplastics into particulate conversion, unable to preserve their original morphology. Additionally, excessive introduction of mechanical structures causes high recovery rate losses when collecting tissue homogenate and may promote fragmented single-type nanoscale plastics to reaggregate into mixed-type microplastics due to frequent shear stress. Microplastic morphology and composition after pretreatment maintain large differences from in-situ environments, affecting subsequent external characterization statistical results of microplastics in organisms; \textcircled{3} Using alkaline digestion solutions or single enzyme solutions to digest biological tissues to obtain separated microplastics. Individual use of alkaline digestion solutions and their combined digestion solutions will dissolve specific types of microplastics. For example, 10% KOH solution corrodes PC and PET high molecular weight polymers, and 10-20% NaOH solution similarly degrades PET, PU, and PC microplastics. According to existing experimental data from methane seep seawater microplastic extraction, bottom seawater deposited in cold seep fluids is rich in various difficult-to-degrade, corrosion-resistant microplastics such as PU, PA, PET, PE. Strong alkaline digestion solutions used for extracting biological tissue microplastics in ordinary aquatic environments or sea areas are not applicable in deep-sea extreme environments and add serious plastic loss risks. Additionally, using single enzyme solutions can selectively decompose cell membranes and protein support structures of biological tissues, extracting tissue microplastics with minimal damage, but slow enzymatic reactions cause excessively long cell digestion times, indirectly increasing experimental costs and hindering widespread application. Bivalves in methane seeps possess harder and more structurally complex cellular protective architectures, requiring introduction of measures to increase reaction rates to accelerate enzymatic reactions; \textcircled{4} No comprehensive extraction schemes for microplastic size ranges exist. Previous literature only used one or two spectroscopic instruments to identify extracted suspicious microplastics. These spectroscopic instruments have fixed advantageous microplastic identification ranges, such as FTIR detection limits of 200 μm and Raman focusing on extremely small-size microplastic identification of <20 μm. Due to limitations from few detection methods, comprehensive microplastic distribution ranges cannot be precisely obtained, so size-based extraction is almost never performed independently. When facing special habitats like methane seeps, microplastic distribution ranges in organisms are relatively broad, mainly concentrated within 0.1-100 μm, but small amounts of extremely large-size microplastics>1 mm also exist. Following previous single-instrument identification can only clarify microplastics within partial advantageous identification sizes. Therefore, to improve overall recovery rates of biological tissue microplastics in deep-sea extreme environments, establishing multi-stage extraction schemes with appropriate steps for size ranges to maximize recovery of microplastics within various size ranges has become an urgent priority.

### SUMMARY

The present application solves problems existing in the prior art and provides a size-based hierarchical extraction and identification method for microplastics in bivalves from deep-sea methane seeps. The method proposed by the present application can ignore cellular structural enhancement imposed by extreme environments, achieving the precision of non-destructive digestion and extraction of full-scale microplastics within tissues. Additionally, microplastics of various size ranges can be separated, and corresponding spectroscopic instruments with advantageous identification capabilities can be customized, reducing degradation and fragmentation of microplastics during extraction and identification processes, while considering future microplastic aging test steps to achieve the requirements for non-destructive detection of biological microplastic adsorption in deep-sea extreme environments, providing support for experimental technical methods for research on identifying interaction relationships between deep-sea environmental ecosystems and seafloor indicator organisms, as well as between biological life activities of organisms and microplastics.

The objective of the present application is to provide a size-based hierarchical extraction and identification method for microplastics in bivalves from deep-sea methane seeps, comprising the following steps:
S1. Frozen preservation: Freeze retrieved bivalves for preservation;
S2. Tissue pretreatment: After thawing the bivalves frozen for preservation in step S1 undergo constant temperature thawing and closed dissection, obtain various biological tissues of the bivalves, and freeze-dry the various biological tissues of the bivalves for later use;
S3. Enzyme solution pre-digestion: add trypsin solution for digestion to the various biological tissues of bivalves obtained in step S2 to perform digestion, to obtain a primary enzyme digestion solution with numerous tissue filamentous condensates;
S4. pH phased enhancement: Use pH adjustment solution to adjust the pH of the primary enzyme digestion solution obtained in step S3 to 7.5, then add trypsin solution to the enzyme digestion solution multiple times to accelerate the breakdown of filamentous condensates, to obtain a secondary enzyme digestion solution, the volume of trypsin solution added each time being ⅓ to ¼ of the volume of the trypsin solution for digestion in step S3;
S5. 30% hydrogen peroxide digestion: Pick out the filamentous condensates from the secondary enzyme digestion solution obtained in step S4, add 30% hydrogen peroxide solution by mass percentage to the filamentous condensates, and digest the remaining cellular adhesive materials at 60-65° C. for 2-4 hours until no bubbles are generated in the digestion solution, i.e., complete digestion is achieved to obtain a nearly transparent solution with no obvious suspended matter, to obtain a hydrogen peroxide digestion solution;
S6. Mixed digestion solution size-based extraction: Mix the secondary enzyme digestion solution obtained in step S4 and the hydrogen peroxide digestion solution obtained in step S5, perform first-size extraction to enrich all target objects>10 μm onto a filter membrane, to obtain a first-size extraction filter membrane; then perform second-size extraction on the first-size filtrate to enrich all target objects of 1-10 μm onto a filter membrane, to obtain a second-size extraction filter membrane; then perform third-size extraction on the second-size filtrate to enrich all target objects of 0.1-1 μm onto a filter membrane, to obtain a third-size extraction filter membrane;
S7. Subject microplastics on the first-size extraction filter membrane, second-size extraction filter membrane, and third-size extraction filter membrane obtained in step S6 to characteristic observation, microscopic imaging, and compositional spectral detection in sequence to obtain size-based abundance information of full-scale microplastics in various biological tissues in the bivalves.

To obtain size-based variation characteristics of microplastics in cold seep indicator organisms from methane seeps, the present application adopts a pH phased enhancement enzyme-hydrogen peroxide mixed digestion method combined with a hierarchical extraction protocol to construct a complete extraction method applicable to microplastics in bivalves under deep-sea extreme environments. This method targets the morphological characteristics of bivalves in methane seeps, with self-prepared seawater approximating cold seep fluid properties to replace washing solutions that alter osmotic pressure differences, abandoning cumbersome and loss-prone mechanical homogenization steps, using enhanced biochemical reaction complete digestion protocols to replace traditional semi-digestion steps. The improved extraction protocol can completely release microplastics from complex organisms while reducing microplastic degradation effects caused by chemical reagents, simultaneously combining size-based extraction methods in filtration operations with advantageous regions of various identification instruments, optimizing methods for extracting full-scale microplastics from bivalves in extreme environments and their supporting identification protocols.

Compared to disadvantages of extraction methods in the prior art based on ordinary aquatic environments involving ultrapure water/organic solvent cleaning of bivalve tissues, mechanical grinding to obtain tissue homogenate, and alkaline digestion/single enzyme solution digestion of tissue cells, the present application starts from morphological characteristics of bivalves nurtured in methane seeps, completing comprehensive optimization adjustments in preservation and cleaning, tissue pretreatment, digestion processes, and filtration extraction. According to advantageous identification intervals of existing various identification instruments, size-based extraction of microplastics is performed to display distribution intervals of full-scale microplastics contained in organisms under special habitats. The objectives are to supplement basic research on microplastics present in deep-sea extreme environments being ingested by bivalves occupying ecological niches, and reduce microplastic loss, fragmentation damage, and dissolution effects caused by biological tissue microplastic extraction protocols under ordinary aquatic environments, guaranteeing as much as possible the consistency of morphology of extracted microplastics with occurrence states in in-situ environments, while adding microplastic size-based extraction steps to obtain pathway characterization of microplastic migration and digestion states within bivalves. The size-based hierarchical extraction and identification method for microplastics proposed by the present application can effectively explore feeding efficiency of bivalves when cold seep fluids gush in methane seeps. Microplastic abundance present in various tissue sites obtained through this method can simulate restored microplastic circulation routes in the body, to understand their caused biological damage effects, and further clarify spatial rates of microplastic absorption by deep-sea extreme ecosystems. This is an integrated detection-identification method for full-scale capture and size-based extraction of microplastics covering 0.0001-5 mm in biological tissues.

Preferably, the specific steps of step S1 are: Select undamaged bivalves, wash the bivalves multiple times until adsorbed mud on the surface is removed, then place the cleaned bivalves into sterile containers and preserve frozen at −20° C. The bivalves proposed by the present application comprise mussel beds, vesicomyid beds, white clam beds, etc.

Still more preferably, the specific steps of step S1 are:
S11. Sampling: Use a television grab on a research vessel to sample located bivalve beds in cold seep areas (such as mussel beds, vesicomyid beds, white clam beds, etc.);
S12. Seawater cleaning: Use a ship-mounted seawater circulation system to draw surface seawater, select intact and undamaged bivalves, and clean the retrieved bivalves multiple times until sediment mud adsorbed on surfaces is removed;
S13. Frozen preservation: Place the cleaned bivalves in sterile self-sealing bags, and after recording serial numbers, preserve the bivalves frozen in a −20° C. freezer.

Preferably, the specific steps of step S2 are: Thaw the bivalves frozen and stored in step S1 at a constant temperature of 4° C. for 0.5-1.5 h, then after closed dissection, obtain various biological tissues of the bivalves; wash the various biological tissues of the bivalve mollusks with washing solution to remove seawater microplastics and various impurities attached outside the bivalve tissue cells, then freeze and preserve them at −80° C. for 5-7 h, and freeze-dry at −60° C. for 20-28 h for later use.

More preferably, the washing solution is prepared by the following steps: Add 5.04 g sodium chloride powder, 0.06 g barium chloride powder, 0.12 g ferrous sulfate powder, 0.02 g manganese sulfate powder, 1.2 g magnesium chloride powder, 0.4 g potassium chloride powder, 0.4 g calcium chloride powder, 2.6 mg sodium nitrate powder, 22 mg sodium silicate powder, 1 mg sodium dihydrogen phosphate powder, and 2.5 g sodium sulfate powder to 800 mL of ultrapure water, dropwise add 23.42 mL of concentrated hydrochloric acid while continuously shaking and stirring, then dilute to 1 L and filter several times using 0.1 μm aqueous microporous filter membrane to obtain the washing solution.

Still more preferably, the specific steps of step S2 are:
S21. Constant temperature thawing: Select required bivalves for detection from the −20° C. freezer in advance and place them in a 4° C. refrigerator to thaw for 1 h;
S22. Equipment preparation: Before dissection experiments, all scissors, tweezers, aluminum boxes, and carriers need to undergo ultrasonic oscillation for 30 min in a sonication cleaning machine filled with ultrapure water filtered through a 0.1 μm aqueous microporous filter membrane. Before dissection begins, rinse the equipment and aluminum foil multiple times with the above filtered ultrapure water for later use;
S23. Closed dissection: Cover the dissection table with aluminum foil rinsed multiple times with the ultrapure water, with the entire dissection table in a closed ventilation room. Take whole bivalve samples, use a dissection knife to cut the adductor muscles close to the inner shell surface to completely display internal tissues, and use spring scissors to carefully cut out various biological tissues of the bivalves (visceral mass, gills, lips, intestines, foot, adductor muscles, mantle). The dissected tissues are to be cleaned. After cleaning, place each tissue in a 60 mm custom aluminum box for freeze-drying, and make records;
S24. Prepare washing solution approximating cold seep fluid: Sequentially weigh 5.04 g sodium chloride powder (AR), 0.06 g barium chloride powder (AR), 0.12 g ferrous sulfate powder (AR), 0.02 g manganese sulfate powder (AR), 1.2 g magnesium chloride powder (AR), 0.4 g potassium chloride powder (AR), 0.4 g calcium chloride powder (AR), 2.6 mg sodium nitrate powder (AR), 22 mg sodium silicate powder (AR), 1 mg sodium dihydrogen phosphate powder (AR), and 2.5 g sodium sulfate powder (AR), add them to 800 mL ultrapure water, dropwise add 23.42 mL concentrated hydrochloric acid (12 mol/L, AR) while continuously oscillating and stirring, then dilute to 1 L, filter three times with a 0.1 μm aqueous microporous filter membrane, set aside the filtered washing solution for later use;
S25. Cleaning and sample preparation: Rinse the dissected biological tissues with filtered washing solution to remove seawater microplastics and various impurities attached outside the bivalve tissue cells, and after rinsing three times, let them stand to drain excess water, and place them in 60 mm aluminum boxes for later use;
S26. Weigh wet weight: Place each aluminum box containing bivalve tissues on the electronic analytical balance, weigh the wet weight of the tissues in tare mode, and record the data;
S27. Frozen preservation: Place each weighed aluminum box in a −80° C. medical freezer for 6 h, and attach the wet weight labels;
S28. Freeze-drying: Slightly open each stored aluminum box, place them in a freeze dryer at −60° C., and freeze-dry for 24 h;
S29. Weigh dry weight: Place each freeze-dried bivalve tissue on the analytical balance, weigh the dry weight, and record the data.

Freeze-drying pretreatment removes water from tissues in vapor form, preserving the microplastic morphology therein, reducing microplastic loss in samples, and obtaining the net dry weight of samples, while also improving subsequent rates of cell membrane and organic matter digestion.

The prior art remains confined to various reagent optimization extraction steps for bivalves bred in ordinary aquatic environments such as shallow seas, rivers, and lakes, rarely involving explanation and discussion of bivalve microplastic size-based extraction protocols, and more lacking in removing digestion efficiency limitations of strengthened cellular connective tissues and enhanced extracellular matrices in bivalves by deep-sea extreme environments. Deep-sea extreme environment bivalve sample preservation and adaptive cleaning are prerequisites for measuring correctness of microplastic extraction experiments. As seafloor indicator organisms in methane seeps, bivalves continuously ingest surrounding microplastics resuspended in surrounding seawater and sediments from the very beginning of their existence. These microplastics, having experienced methane gas etching from decomposition of hydrates buried in rock layers, are more easily fragmented into small-size range microplastics or even nanoplastics. Through the full-scale microplastic size-based extraction protocol of the present application, understanding the long-term dynamic accumulation situations of microplastics within bivalves, assessing the extent of microplastic pollution in deep-sea extreme environments, and identifying the extent of accumulation of microplastics enriched in extreme environments within centennial scales and their migration and transformation processes in oceans have important significance.

The present application reconstructs cold seep fluid solution properties adapted to bivalve survival and uses them in preservation and cleaning steps, reducing cellular rupture and swelling phenomena occurring during strong osmotic pressure imbalances caused by inappropriate salt solutions or ultrapure water; while abandoning mechanical grinding homogenization operations used in conventional literature, simple and effective freeze-drying combined with enhanced enzyme combination digestion solutions sufficiently guarantee in-situ preservation of ultra-large size microplastics adsorbed by tissue cells without loss; using pH phased enhancement enzyme solutions for pre-digestion of large tissue condensates to remove limitations of connective tissues and intercellular matrix connections strengthened by high-pressure environments, with hydrogen peroxide serving as the final fragmenter of filamentous condensates to maximally restore microplastic types and apparent morphology adsorbed by bivalves in in-situ environments, achieving complete reproduction of original conditions of seafloor bivalve microplastic pollution; the pioneered full-scale microplastic size-based extraction protocols of biological tissues combined with advantageous identification intervals of various identification instruments cancels succession relationships between instruments, drives all suspected microplastics to begin detection in multiple classified channels at the same stage, greatly reducing instrument time costs.

The present application aims to explore microplastic accumulation abundance in special microplastic-accumulating indicator organisms represented by bivalves in methane seeps in deep-sea extreme environments and microplastic migration routes under life activity stress. Through group-retrieval of seafloor bivalve beds coupled with survival timelines of individual bivalves, extreme ecosystem microplastic adsorption rates over time can be further calculated, clarifying the degree of enrichment of methane seeps as microplastic hotspot regions. All exploration beginnings stem from the full-scale microplastic hierarchical extraction and identification integrated protocol proposed by the present application. The present application supplements existing technology gaps regarding bivalve research under extreme environments and maintains original bivalve cellular states through self-prepared cold flow fluid methods. This is a field not yet carefully studied by scholars, providing effective research protocols for accurately quantifying human activity damage effects on deep-sea extreme systems, providing guidance and technical reference for microplastic extraction protocols of indicator organisms in other extreme sea areas.

Preferably, the trypsin solution in step S3 or S4 is prepared by the following steps: Weigh 6.80 g potassium dihydrogen phosphate, add 500 mL water to dissolve it, adjust pH to 7.5 with 0.1 mol/L potassium hydroxide solution, add 30.00 g trypsin, dissolve with water, then dilute to 1 L to obtain the trypsin solution.

Preferably, the specific steps of step S3 are: Add trypsin solution for digestion to the various biological tissues of bivalves obtained in step S2 at a ratio of 1 g biological tissue dry weight to 30-40 mL trypsin solution, and digest at 35° C.-40° C. for 24-30 h to obtain a primary enzyme digestion solution with numerous tissue filamentous condensates.

Further preferably, the specific steps of step S3 are:
S31. Prepare trypsin solution: Weigh 6.80 g potassium dihydrogen phosphate, add 500 mL water to dissolve, adjust pH to 7.5 with 0.1 mol/L potassium hydroxide solution, add 30.00 g trypsin, dissolve with water, then dilute to 1 L. Filter three times using a 0.1 μm polyvinylidene fluoride filter membrane, and store in a −20° C. freezer for later use;
S32. Constant temperature digestion: According to the tissue dry weight, place each freeze-dried tissue in a cleaned 50 mL or 100 mL stoppered ground-mouth conical flask, add a certain amount of trypsin solution for digestion at a ratio of 1 g tissue dry weight to 30-40 mL trypsin solution, digest at 35° C.-40° C. on a constant temperature shaker at 100 rpm for 24-30 h, removing every 4 h and placing under magnetic stirring for accelerated mixing, to finally obtain a primary enzyme digestion solution with numerous tissue filamentous condensates. At this point, most bivalve tissues are enzyme-ruptured, and microplastics are released into the digestion solution, but some microplastics are still adhered to filamentous condensates and remain unseparated.

Preferably, the pH adjustment solution in step S4 is potassium dihydrogen phosphate solution with a mass concentration of 0.0136 g/mL or potassium hydroxide solution with a mass concentration of 0.0562 g/mL.

More preferably, the specific steps of step S4 are:
S41. Prepare pH adjustment solution:
\textcircled{1} Weigh 0.68 g potassium dihydrogen phosphate, add 50 mL water to dissolve, and bottle for later use after oscillating;
\textcircled{2} Weigh 2.81 g potassium hydroxide, add 50 mL water to dissolve, and bottle for later use after oscillating; The above solutions need to be filtered three times through a 47 mm, 0.1 μm polyvinylidene fluoride filter membrane before use.
S42. Adjust pH of the primary enzyme digestion solution: After constant temperature digestion, take out the conical flasks every 12 h, measure the acidity-alkalinity with a pH meter, use a 10 mL glass syringe to draw pH adjustment solution, and add dropwise with shaking to maintain the primary enzyme digestion solution pH at 7.5;
S43. Secondary enzyme digestion: Add ⅓-¼ volume of trypsin solution used for digestion in step S3 to repeatedly rinse the pH meter and accelerate filamentous condensate breakdown. Repeat 4 times total for each sample group, obtaining secondary enzyme digestion solution.

Preferably, in step S6, the first-size extraction uses a 50 mm diameter glass filter core paired with a 47 mm diameter, 1200 mesh stainless steel membrane; the second-size extraction uses a 25 mm diameter filter core paired with a 25 mm diameter, 1 μm pore size glass microfiber filter membrane; the third-size extraction uses a 25 mm diameter filter core paired with a 25 mm diameter, 0.1 μm pore size inorganic aluminum oxide membrane.

More preferably, the specific steps of step S6 are:
S61. First-size (>10 μm) extraction: Select a 1 L glass vacuum suction filtration device paired with a 50 mm diameter glass filter core for first-size suction filtration, using a self-cut 47 mm diameter, 1200 mesh stainless steel membrane as filter. At experiment start, pre-clean the device multiple times with ultrapure water filtered through a 0.1 μm aqueous microporous filter membrane, then add secondary enzyme digestion solution and hydrogen peroxide digestion solution together into the device, and repeat vacuum suction filtration steps three times, using the previous filtrate as rinse solution each time. Before completing filtration, repeatedly rinse the interior walls of the filter and conical flasks with the filtered ultrapure water to concentrate all target objects>10 μm onto the filter membrane. Place the purification filter membrane in a 60 mm aluminum box and dry in the oven at 60° C. for 3 h, and store the first-size extraction filter membrane at room temperature; the filtrate in the filter bottle will be transferred;
S62. Second-size (1-10 μm) extraction: Rinse a 250 mL micro sand core filtration device with filtered ultrapure water, the device using a 25 mm diameter filter core paired with a 25 mm, 1 μm glass microfiber filter membrane. After cleaning, pour the first-size filtrate to be transferred into the filter cup for vacuum suction filtration, and repeat suction filtration three times, using the obtained filtrate as rinse solution each time. Before completing filtration, repeatedly rinse the interior walls of the filter cup and the interior walls of the 1 L filter bottle with filtered ultrapure water to ensure collection of all particles. Place the purification filter membrane in a 30 mm aluminum box and dry in the oven at 60° C. for 3 h, and store the dried second-size extraction filter membrane at room temperature;
S63. Third-size (0.1-1 μm) extraction: The third-size extraction steps are the same as the second-size, only replacing the glass microfiber filter membrane with a 25 mm diameter, 0.1 μm pore size inorganic aluminum oxide membrane. When completing filtration, repeatedly rinse the interior walls of the filter bottle and filter cup with small amounts of filtered ethanol solution, and filter the cleaning solution. Because the obtained filter membrane has a relatively fragile support ring and membrane surface, place it in a 30 mm glass Petri dish and dry in the oven for 3 h, then store at room temperature for instrument identification;
S64. Extraction: Place the first-size extraction filter membrane face-up in a 50 mL beaker, add approximately 15 mL anhydrous ethanol solution until the liquid surface completely submerges the filter membrane surface, seal the beaker with cleaned aluminum foil fixed with a rubber band, and place in a constant temperature shaker and shake at room temperature at 100 rpm for 12 h, completely releasing sediment particles and suspected microplastic particles attached to the filter membrane into solution;
S65. Ethanol rinsing: Remove the extracted first-size extraction filter membrane, and repeatedly rinse the front and back surfaces and membrane edge gaps with anhydrous ethanol until no obvious particles remain on the filter membrane;
S66. Filtration and membrane preparation: Re-vacuum filter the anhydrous ethanol rinse solution, and the filtration steps are the same as for the first-size extraction filtration, replacing the filter membrane with a 47 mm, 10 μm mixed cellulose filter membrane; repeat three times, finally cleaning the cup walls and containers multiple times with anhydrous ethanol to concentrate all suspected microplastics onto the membrane;
S67. Constant temperature drying: Place the obtained first-size microplastic purification filter membrane in a 60 mm aluminum box, dry in the oven at 60° C. for 2 h, and prepare for the next step microplastic identification.

All ultrapure water, prepared solutions, reagents, and digestion solutions mentioned above must be pre-filtered through a 0.1 μm aqueous microporous filter membrane and a 0.1 μm polyvinylidene fluoride filter membrane, respectively, to remove inherent impurities in the prepared solutions. Similarly, before using glassware, beakers, tweezers, scissors, and other equipment, they must be soaked in an ultrasonic bath containing filtered ultrapure water for 30 minutes to eliminate potential impurity interference. Before using filter membranes, carefully use tweezers to immerse the membrane in anhydrous ethanol, washing back and forth, and finally rinsing the membranes with a small amount of anhydrous ethanol.

Compared with the processes of extracting microplastics from shallow-water bivalve samples in the prior art, the present application avoids destroying the inherent microplastic morphology in tissues by preparing a cleaning solution that is close to the in-situ living environment and using a non-homogeneous operation method. Furthermore, by innovatively using a double mixed solution of a pH phased enhancement trypsin solution and 30% hydrogen peroxide to digest biological tissues, this combined digestion solution only produces a large amount of oily debris foam, has no negative impact on almost all types of microplastics, has high fidelity, and can extract microplastics present in the organism without damage to the greatest extent. At the same time, the use of a size-based hierarchical extraction protocol instead of the traditional density flotation method to separate microplastics in the concentrated solution can completely avoid the problems of insufficient extraction efficiency of the flotation agent, high cost, and erroneous digestion, and facilitates the subsequent combined identification instruments to simultaneously perform point-to-point scanning and detection of microplastics, reducing time costs.

Preferably, step S7 specifically comprises the following steps:
S71. Separate the first-size extraction filter membrane into a large-size microplastic filter membrane with particle size greater than 500 μm and a medium-size filter membrane with particle size of 10-500 μm; perform infrared imaging on microplastics in the large-size microplastic filter membrane with particle size greater than 500 μm and qualitatively analyze their distribution to obtain the compositional type uniformity of all suspected microplastics and determine the types of these polymers; perform microscopic infrared imaging on microplastics in the medium-size filter membrane with particle size of 10-500 μm and qualitatively analyze their composition, and calculate their technical parameters including particle count, particle diameter, and equivalent area;
S72. Perform morphological observation, microscopic imaging, and compositional analysis on small-size microplastics with particle size of 1-10 μm in the second-size extraction filter membrane, count the size, color, shape, equivalent diameter, and particle count of all small-size microplastics, and perform Raman imaging to obtain compositional distribution information;
S73. Perform morphological observation, microscopic imaging, and compositional analysis on submicron-size microplastics with particle size of 0.1-1 μm in the third-size extraction filter membrane, count the size, color, shape, and particle count of all submicron-size microplastics, and perform microscopic imaging to obtain compositional distribution information.

More preferably, step S7 further comprises: S74. Extract the microplastics of each size separately, and use ATR-FTIR, LDIR, Raman, and micro-Raman spectrometers; the total measured microplastics represent the full-scale microplastic content of a specific tissue in a single bivalve; combined with abundance correction in the experimental steps, obtain size-based abundance information of full-scale microplastics in various biological tissues within bivalve mollusks.

The main objective of step S7 is to sequentially identify all full-scale microplastics ranging from 0.0001 to 5 mm on the three microplastic purification filter membranes. Based on the above hierarchical extraction protocol, purification filter membranes can be obtained for large-size microplastics (>10 μm), small-size microplastics (1-10 μm), and submicron-size microplastics (0.1-1 μm). To obtain the morphological characteristics, microscopic morphology imaging, and compositional spectral data of the purified microplastics in each size range, specific detection steps for the three size ranges of microplastic purification filter membranes are as follows:

(1) Large-size microplastic morphological observation (>10 μm): The main objective of this step is to divide the broad detection range of large-size microplastics (>10 μm) into the two purification filter membranes of large-size microplastics (>500 μm) and medium-size microplastics (10-500 μm), and record the morphological characteristics of these two size distributions, including macroscopic information such as color, shape, size, and quantity, to facilitate subsequent synchronous scanning by advantageous identification instruments.

Place the first-size microplastic purification filter membrane from step S67 of the size-based hierarchical extraction protocol after constant temperature drying under an optical microscope for morphological observation and quantity statistics. The observation instrument for this step is not unique; for example, a high-resolution inverted fluorescence microscope with a 4× objective lens or a stereomicroscope also with a 4× objective lens can be used. Using a stereomicroscope as an example, observe the membrane surface in a “Z” pattern, record the morphology and color of all suspected microplastics on the membrane, search for microplastics larger than 500 μm in the field of view, capture photographs, measure their sizes, and pick them all out to transfer to a new mixed cellulose filter membrane with proper labeling. Thus, the obtained >500 μm purification filter membrane achieves re-extraction of the first-size purification filter membrane in the identification protocol.

(2) Microscopic imaging and compositional spectroscopy of the re-extracted filter membrane of the first-size purification filter membrane (>500 μm): The main objective of this step is to perform infrared imaging on the selected >500 μm microplastics and qualitatively analyze their distribution, obtaining the compositional uniformity of the entire suspected microplastics and determining the types of these polymers. Specific steps are as follows:

Observe all selected microplastics on the >500 μm purification filter membrane, and select one typical microplastic with highly characteristic features for infrared imaging and identification of its compositional distribution regions. The observation instrument for this step is not unique; instruments can include FTIR, ATR-FTIR, or FTIR with focal plane array detector (FPA). Using ATR-FTIR as an example, after background correction, place the selected typical microplastic on the ATR crystal surface, apply pressure to ensure tight contact between the sample and the ATR crystal, start the FTIR spectrometer, and perform spectral scanning. Select a wavenumber range of 4000 to 400 cm⁻¹ to cover characteristic absorption peaks of most polymers, with each scan repeated three times. Fit the characteristic peaks. A spectral library match greater than 70% indicates microplastic. Then, set the infrared imaging parameters of the FTIR microscope, and select an imaging area matching the microplastic size, with a spatial resolution of 5 μm, spectral resolution of 8 cm⁻¹, and 30 seconds per scan, generating infrared images for constructing chemical composition distribution maps.

(3) Microscopic imaging and compositional spectroscopy of the first-size purification filter membrane (10-500 μm): The main objective of this step is to perform microscopic infrared imaging and qualitative compositional analysis on the remaining suspected microplastics on the first-size purification filter membrane after re-extraction, calculating parameters such as particle number, particle diameter, and equivalent area. The detection instruments for this size range are not unique; laser infrared spectrometers or μFTIR can be used. Using laser infrared spectrometer (Agilent 8700 LDIR) as an example, specific steps are as follows:

Place the re-extracted first-size purification filter membrane in a 50 mL beaker, immerse the membrane surface again with anhydrous ethanol, shake at 100 rpm for 12 hours on a shaker, wash both sides and edges of the membrane multiple times, place the extraction solution on a constant temperature heating plate at 60° C. until concentrated to a 100 μL suspension, and add the suspension dropwise onto a cleaned high-reflective glass. After complete ethanol evaporation, identify 10-500 μm microplastics under the laser infrared spectrometer with the match degree set as >0.7, each scan repeated three times, to obtain infrared images and polymer types of all microplastic particles on the filter membrane.

(4) Small-size microplastic morphological observation, microscopic imaging, and compositional spectroscopy (1-10 μm): The main objective of this step is to perform morphological observation, microscopic imaging, and compositional analysis on small-size microplastics (1-10 μm) on the second-size purification filter membrane, calculating size, color, shape, equivalent diameter, and particle number of all small-size microplastics, and perform Raman imaging to obtain compositional distribution information. The instrument selection for this step is not unique; using HORIBA LabRAM Odyssey high-resolution Raman spectrometer as an example, specific steps are as follows:

Place the second-size purification filter membrane on the stage of the Raman spectrometer, and select 785 nm excitation wavelength, with laser power of 100 mW, optical resolution of 1 μm⁻¹, scanning range of 500-3000 cm⁻¹, integration time of 5 s, and number of integrations set to 3. Control the joystick to scan the membrane surface in a “Z” pattern, identify all 1-10 μm suspected microplastics on the membrane, and record morphological information including type, color, shape, size of each, with a spectral library match rate greater than 70% considered as microplastic. Each scan is repeated three times. Then, use the Mapping function for point-to-point area scanning with the same integration time and number of integrations as above, controlling the time within 30 s, to obtain compositional distribution imaging spectra of microplastics on the entire membrane surface.

(5) Submicron-size microplastic morphological observation, microscopic imaging, and compositional spectroscopy (0.1-1 μm): The main objective of this step is to perform morphological observation, microscopic imaging, and compositional analysis on submicron-size microplastics (0.1-1 μm) on the third-size purification filter membrane, calculating size, color, shape, and particle number of all submicron-size microplastics, and perform microscopic imaging to obtain compositional distribution information. The instrument selection for this step is not unique; using Thermo Scientific DXR 3xi Raman imaging microscope as an example, specific steps are as follows:

Carefully transfer the filter membrane to the microscope stage using microscopic tweezers, calibrate the inorganic aluminum oxide filter membrane background, and set Raman microscope parameters with laser power of 5 mW, spectral range of 500-4000 cm⁻¹, and integration time of 1 s. Use surface scanning mode to automatically locate particle imaging areas and automatically stitch each scanning area into a complete Raman image approximating the entire filter membrane surface while performing point spectroscopy. Search for microplastic particles with >70% match rate in the spectral database, switch to the optical module, and record the size, color, type, shape, and other information of each microplastic particle sequentially.

(6) Abundance correction: By extracting microplastics of each size grade separately and pairing with advantageous identification spectroscopic instruments, the problem of multiple identifications due to overlapping identification intervals can be avoided. Therefore, the microplastic content in individual bivalves obtained by this integrated extraction-identification protocol can be converted to detection by four advantageous identification instruments for three size-level purification filter membranes. In this protocol, the sum of microplastics measured by ATR-FTIR, LDIR, Raman, and micro-Raman spectrometers represents the full-scale microplastic content of specific tissues in individual bivalves. More importantly, if the anhydrous ethanol suspension added in the extraction step of identification protocol (3) is less than 100 μL, the final measured content needs to be multiplied by a volume coefficient. If only half the membrane surface area is observed during membrane observation, all subsequently read microplastic morphological information needs to consider the area coefficient.

Compared to the prior art, the present application has the following advantages:

1. Compared to existing microplastic extraction techniques from bivalve biological tissues in shallow-sea or freshwater ordinary aquatic environments, the integrated size-based extraction and identification method proposed by the present application corrects conventional ultrapure water or organic preservation and washing solutions used for preserving bivalve tissues in ordinary aquatic environments by formulating self-made washing supplemental solutions with cold seep fluid properties to balance enormous biological osmotic pressure differences and maintain normal cell morphology, preventing premature escape of microplastics during long-term storage. Additionally, conventional techniques use grinding/mechanical homogenization plus alkaline digestion solutions and unenhanced enzyme solutions to achieve complete digestion of marine biological tissues. However, the deep-sea high-pressure extreme environment creates stronger muscle fiber structures and antioxidant-resistant membrane protein structures in seafloor bivalves. Intense mechanical impeller rotation cuts intercellular connections while continuously crushing large-sized microplastics. Ordinary alkaline digestion solutions cause chemical damage to microplastics by hydrolyzing ester and amide groups in main chains when dissolving cell membranes. The present application abandons mechanical tissue homogenization to preserve large microplastic morphology and uses pH-enhanced enzyme solutions to maintain high degradation rates of freeze-dried tissues during pre-digestion, with hydrogen peroxide solution completing final elimination of remaining filamentous condensates. This chemically enhanced catalytic cell rupture digestion method completely preserves original microplastic morphology, eliminates degradation effects of ordinary alkaline digestion solutions on specific microplastics, and efficiently breaks down environmentally adapted muscle fiber tissues to expose all microplastics encapsulated by tissue cells.

2. Different from single identification instruments mentioned in the prior art, the present application uses size-based hierarchical filtering to obtain three purification filter membranes paired with corresponding advantageous identification instrument combinations, enabling collection of multi-dimensional morphological information including size, particle diameter, color, type, and shape for full-scale microplastics (0.0001-5 mm), expanding traditional abundance statistical intervals and microplastic research intervals limited by instrument detection limits. The present application is the first to incorporate bivalves from methane seep areas into the research process of microplastic extraction from biological tissues, optimizing and adapting the pretreatment, digestion, purification, and identification processes, creating experimental protocols specifically for microplastic extraction from organisms in deep-sea extreme environments, thereby revealing the final migration destinations of long-term adsorbed microplastics in indicator organisms from seafloor methane seeps, filling gaps in this field.

3. The present application is simple, easy to operate, and reproducible, suitable for exploring characterization methods of microplastic accumulation in various tissues of bivalves in deep-sea extreme ecosystems. Combined with the retrieved seafloor bivalve bed layers, it can extrapolate to the temporal course and historical accumulation of microplastic absorption in entire extreme ecosystems, solving the challenge of large-scale sampling, extraction, and analysis in deep-sea extreme environments, providing scientific testing strategies for assessing the historical impact of microplastic pollution in specific marine areas.

### BRIEF DESCRIPTION OF THE DRAWINGS

FIGURE is a process flow chart of the method proposed in the present application.

### DETAILED DESCRIPTION OF THE EMBODIMENTS

The following embodiments are further descriptions of the present application, and not limitations of the present application.

Unless otherwise defined, all technical terms used below have the same meaning as usually understood by those of ordinary skill in the art. The technical terms used herein are only for the purpose of describing specific embodiments, and are not intended to limit the scope of protection of the present application. Unless otherwise specified, the experimental materials and reagents in this document are all conventional commercially available products in this technical field.

The present application uses the deep-sea extreme environment of the South China Sea—methane seeps at the active cold seep of Haima—as a research sea area, and selects one of its seafloor filter-feeding indicator organisms—Gigantidas haimaensis (hereinafter referred to as mussel)—as a case demonstration. The technical flowchart of the process of extracting microplastics from methane seep bivalves is as shown in the FIGURE, and includes the following steps:

\textcircled{1} On the scientific research vessel, use a Television grab to sample the mussel target bed.

\textcircled{2} Use the shipborne seawater circulation system to wash mud off the surface of the mussels, and pack the mussels at −20° C. for preservation.

\textcircled{3} Select target mussels and thaw at 4° C., wash all microscopic operation tools, and on a closed ventilated dissection table carefully dissect out the gills, mantle, labial palps, foot, intestine, visceral mass, and adductor muscle in sequence, and place them into clean 60 mm aluminum boxes.

\textcircled{4} Prepare a washing solution similar to cold seep fluid, filter it, and continuously wash the dissected biological tissues.

\textcircled{5} Weigh the wet weight of the biological tissues, and after −80° C. freeze molding, place them in a freeze dryer for freeze-drying, and weigh the dry weight.

\textcircled{6} Prepare an enzyme solution, add a certain amount of enzyme solution according to a certain mussel dry weight ratio, digest 24 h at 37° C. on a constant-temperature shaker, and during this period, take out every 4 h and stir evenly under a magnetic stirrer.

\textcircled{7} After constant-temperature digestion, prepare a pH adjustment solution, add the adjustment solution dropwise every 12 h to maintain the pH of the digestion solution at 7.5, and add enzyme solution for secondary digestion of the suspension. This process is repeated 4 times.

\textcircled{8} Pick out undissolved filamentous condensates in the digestion solution, add into filtered hydrogen peroxide solution, and place the whole on an electric heating plate at 60° C. for 3 h for digestion.

\textcircled{9} Subject the obtained enzyme solution and hydrogen peroxide mixed digestion solution to hierarchical vacuum filtration in sequence, repeat each filtration step three times, and finally rinse the glassware with anhydrous ethanol. The filter membranes use a 47 mm diameter, 1200 mesh pore size stainless steel membrane, a 25 mm, 1 μm glass microfiber filter membrane, and a 0.1 μm pore size inorganic alumina membrane. After hierarchical filtration, place respectively into a 60 mm aluminum box, 30 mm aluminum box, and 30 mm glass Petri dish, and dry to obtain extraction filter membranes for the three types of microplastic size collections.

\textcircled{10} Add anhydrous ethanol to the dried first-size extraction filter membrane for extraction, shake for 12 h on a shaker, continuously rinse both sides of the filter membrane with anhydrous ethanol, and then perform vacuum filtration again on the first-size. Select a 47 mm, 10 μm mixed cellulose filter membrane as the filter membrane, and obtain a purified filter membrane after drying.

\textcircled{11} Place the first-size purified filter membrane under a stereomicroscope, scan the membrane surface in a “Z” pattern, record the particle number, morphology, and color of all suspected microplastics on the membrane, search for and pick out suspected microplastics>500 μm in the field of view for transfer to a new mixed cellulose filter membrane, making annotations. Select the most characteristic suspected microplastics on the new membrane for qualitative analysis under ATR-FTIR and infrared imaging. Microplastics have a spectral library similarity>70%. Obtain all polymer types and single-point infrared images of >500 μm microplastics in the first-size purified filter membrane.

\textcircled{12} Immerse the first-size purified filter membrane after picking in anhydrous ethanol, shake for 12 h on a shaker, rinse both sides of the filter membrane multiple times, then concentrate by heating to a 100 μL suspension. Drop 100 μL suspension on a high-reflective glass, air-dry in a fume hood, and identify microplastics on the glass under a laser infrared spectrometer, with the match rate set at >0.7, to obtain polymer types of 10-500 μm microplastics in the first-size purified filter membrane and laser infrared images on the membrane surface.

\textcircled{13} Place the second-size purified filter membrane on the stage of the Raman spectrometer, set the instrument spectrum parameters, scan the membrane surface in a “Z” pattern, and identify all suspected 1-10 μm microplastics. Microplastics have a spectrum library match rate>70%. Remove >10 μm microplastics, record the type, color, morphology, and size data of each microplastic, and use the regional Mapping function to obtain the Raman imaging distribution map of microplastics on the whole membrane surface.

\textcircled{14} Place the third-size purified filter membrane on the stage of the Raman imaging microscope, calibrate the filter membrane background, set Raman identification parameters and select area scan mode, automatically locate particle imaging areas, identify suspected 0.1-1 μm microplastics on the whole membrane surface, and automatically splice into a complete Raman microscopic imaging map. Database match rate>70% is viewed as microplastic particles. Record the size, color, type, and shape of each microplastic particle.

\textcircled{15} Combine abundance correction in the experimental steps to obtain size-based abundance information of all microplastics in each biological tissue of the whole mussel.

The method proposed in the present application uses various experimental steps, including washing and preserving dissected biological tissues with washing solution, freeze-drying the biological tissues to dehydrate, using pH phased enhancement enzyme-hydrogen peroxide mixed digestion solution, hierarchical vacuum filtration, and size-based advantageous identification to non-destructively and categorically extract microplastics contained in bivalves in extreme environments. The objective is to realize size-based quantitative and qualitative analysis of full-scale microplastics (0.0001-5 mm) in bivalves.

The instruments and equipment used in the following embodiments:

Microscissors, stainless steel tweezers, fine-point tweezers, sampling needle, scalpel, surgical dish, spring scissors, medicine spoon, glass rod, 1 L vacuum filtration device, 250 mL miniature sand core filtration device, ultrasonic cleaner, freeze dryer, oven, 0.0001 g electronic analytical balance, graphite electric heating plate, pH meter, magnetic stirrer, constant-temperature shaker, laser infrared spectrometer, Fourier transform attenuated total reflection infrared spectrometer, stereo microscope, high-resolution Raman spectrometer, Raman imaging microscope, television grab, seawater circulation system, −20° C. refrigerator, 4° C. refrigerator, −80° C. medical refrigerator, ventilation room, dissection table.

The reagents and consumables used in the following preferred embodiments:

Potassium hydroxide, anhydrous ethanol solution, potassium dihydrogen phosphate, trypsin, sodium chloride, barium chloride, ferrous sulfate, manganese sulfate, magnesium chloride, potassium chloride, calcium chloride, sodium nitrate, sodium silicate, sodium dihydrogen phosphate, sodium sulfate, concentrated hydrochloric acid, and 30% hydrogen peroxide solution are all conventional commercial products; 25 mm, 1 μm glass microfiber filter membrane, 47 mm, 0.1 μm polyvinylidene fluoride filter membrane, 47 mm, 0.1 μm aqueous microporous filter membrane, 25 mm, 0.1 μm inorganic alumina membrane, 1200 mesh steel membrane, 47 mm, 10 μm mixed cellulose filter membrane, 60 mm aluminum storage box, 30 mm aluminum storage box, 30 mm glass Petri dish, 10 mL glass syringe, tin foil, rubber band, 50 mL beaker, 2 L beaker, 1 L beaker, 50 mL and 100 mL stoppered ground-neck conical flask, sterile self-sealing bag, ultrapure water, high-reflective glass.

### Embodiment 1

The present embodiment provides a size-based hierarchical extraction and identification method for microplastics in bivalves from methane seeps. Samples are taken from mussel beds near small plume vents of strong methane seeps of the Haima cold seep in the South China Sea, and specifically include the following steps:

(1) Sample collection and washing: Use a television grab on the scientific research vessel to sample the mussel beds bred near the central vent of strong methane seeps of the Haima cold seep. After retrieval to the stern deck, use the shipborne seawater circulation system to wash the mussel shells to remove attached mud.

(2) Frozen preservation: Select complete and unbroken mussels after washing, place them separately into sterile self-sealing bags, record serial numbers, and store frozen at −20° C.

(3) Experimental pretreatment:

\textcircled{1} Constant-temperature thawing: Purposefully select mussels with special meaning from the −20° C. refrigerator, and thaw for 1 h in a 4° C. refrigerator.

\textcircled{2} Instrument preparation: Immerse all scissors, tweezers, aluminum boxes, tin foil, scalpel, and other dissection instruments in an ultrasonic cleaner filled with ultrapure water and sonicate for 30 min, and rinse the above dissection instruments and tin foil multiple times with filtered ultrapure water prior to the experiment.

\textcircled{3} Dissection and subdivision: Open the ventilation system in the closed ventilation room, cover the whole dissection table with clean tin foil, take out the mussels, slightly open the shell gap, cut the adductor muscle on one side with a scalpel to fully open the entire internal tissues of the mussel, use spring scissors to carefully cut out each mussel tissue in sequence (gills, mantle, labial palps, foot, intestine, visceral mass, adductor muscle), and temporarily place them into corresponding 60 mm special aluminum boxes.

\textcircled{4} Washing and preservation: Prepare washing solution similar to cold seep fluid and filter it three times with a 0.1 μm aqueous microporous filter membrane. Wash each dissected tissue and aluminum box with the filtered washing solution multiple times. After draining the water, place the tissues into corresponding aluminum boxes for later use.

\textcircled{5} Wet weight measurement: Place the aluminum box of each mussel tissue on the electronic analytical balance, weigh each tissue wet weight in tare mode, and record the data.

\textcircled{6} Freeze-drying preservation: Place the aluminum boxes of the wet-weighed tissues into a −80° C. medical refrigerator and freeze for 6 h, and label the wet weight labels. Then, place each frozen aluminum box into a freeze dryer at −60° C. and freeze-dry for 24 h.

\textcircled{7} Dry weight measurement: Place each freeze-dried mussel tissue on the electronic analytical balance, weigh the dry weight in tare mode, and record the dry weight data.

(4) Enzyme solution pre-digestion: Prepare trypsin solution and filter three times with the 0.1 μm polyvinylidene fluoride filter membrane, add the freeze-dried tissues into washed 50 mL or 100 mL stoppered ground-neck conical flasks according to tissue dry weight, add trypsin solution according to the ratio of 1 g biological tissue dry weight to 35 mL trypsin solution, and seal and place on the constant-temperature shaker and digest at 37° C. at 100 rpm for 24 h, taking out every 4 h to accelerate digestion with magnetic stirring.

(5) pH phased enhancement: Prepare pH adjustment solution and filter it three times with a 0.1 μm polyvinylidene fluoride filter membrane. Take out the tissue digestion solution, measure the pH with the pH meter, add the adjustment solution dropwise to maintain the digestion solution at pH 7.5, then add enzyme digestion solution again to wash the pH meter and accelerate tissue cell decomposition. Repeat this process four times.

(6) Hydrogen peroxide digestion: Pick out filamentous condensates from the enzyme solution after pH enhancement digestion and place them into a new clean conical flask, add a certain amount of 30% hydrogen peroxide solution, wash the tweezers, and place the flask on a graphite electric heating plate at 60° C. and digest for another 3 h to obtain a clear solution with almost no obvious suspended matter.

(7) Mixed digestion solution size-based hierarchical extraction:

\textcircled{1} First-size (>10 μm) extraction: Wash a 1 L glass vacuum filtration device three times with filtered ultrapure water, load a 47 mm diameter, 1200 mesh stainless steel membrane on the 50 mm filter holder, pour secondary enzyme digestion solution and hydrogen peroxide digestion solution into the filter cup, repeat vacuum filtration three times with each rinse solution used as the filtrate obtained from filtration, continuously rinse the filter cup and two conical flasks, and finally rinse residual digestion solution on the cup wall with a small amount of ultrapure water to obtain the extraction filter membrane. Place the 60 mm aluminum box containing the extraction filter membrane into the oven at 60° C. and dry for 3 h.

\textcircled{2} Second-size (1-10 μm) extraction: Wash a 250 mL miniature sand core filtration device three times with filtered ultrapure water, load a 25 mm diameter, 1 μm pore size glass microfiber filter membrane on a 25 mm filter holder, slowly pour the first-size filtrate into the device multiple times for vacuum filtration, filtering the filtrate three times with each rinse solution used as the filtrate obtained from the last filtration and used to wash the 1 L filter flask and filter cup wall, and finally rinse residual digestion solution with a small amount of ultrapure water. Place the obtained purified filter membrane into a 30 mm aluminum box and dry in the oven at 60° C. for 3 h.

\textcircled{3} Third-size (0.1-1 μm) extraction: Same as the second-size extraction step, wash the miniature sand core filtration device with ultrapure water, change the filter membrane to a 25 mm, 0.1 μm inorganic alumina membrane, slowly pour the second-size filtrate into the filter cup multiple times for vacuum filtration, filtering the filtrate three times. Before completing filtration, rinse the inner wall of the filter cup and the 250 mL filter flask with filtered anhydrous ethanol solution to transfer residual microplastics to the membrane, then place the purified filter membrane into a 30 mm glass Petri dish, dry in the oven at 60° C. for 3 h, and preserve at room temperature.

(8) Extraction and ethanol rinsing: Use a certain amount of anhydrous ethanol solution to immerse the membrane surface of the first-size extraction filter membrane, seal with tin foil, and oscillate in a constant-temperature shaker at room temperature at 100 rpm for 12 h. Then remove the filter membrane, and rinse both sides and edges of the membrane continuously with anhydrous ethanol until no obvious particles remain attached.

(9) Vacuum filtration and membrane preparation: Continuously wash the 1 L glass vacuum filtration device with ultrapure water, select a 47 mm, 10 μm mixed cellulose filter membrane, filter the anhydrous ethanol extraction solution three times, and finally rinse the cup wall and container multiple times with anhydrous ethanol. Place the purified first-size filter membrane in a 60 mm aluminum box and dry in the oven at 60° C. for 2 h.

(10) Quality control: All ultrapure water, prepared solutions, reagents, and digestion solutions used above must be separately filtered in advance with a 0.1 μm aqueous microporous filter membrane and a 0.1 μm polyvinylidene fluoride filter membrane to remove impurities existing in the solution itself. Similarly, before using glassware, beakers, tweezers, scissors, and other instruments, all must be immersed in an ultrasonic bath with filtered ultrapure water for 30 min to eliminate potential impurity influences as much as possible. Before using filter membranes, use tweezers to carefully soak the filter membranes in anhydrous ethanol and wash back and forth, finally rinsing with a small amount of anhydrous ethanol. During the experiment, wear cotton gloves and cotton laboratory clothes. Under the same conditions, set a blank control group, with all operations consistent with the experimental group.

(11) Large-size microplastic characteristic observation, infrared imaging, and component spectrum (>500 μm): Place the first-size purified filter membrane obtained from step (9) on the stage of the stereomicroscope, observe the membrane surface in a “Z” pattern, record the morphology and color information of all suspected microplastics (>10 μm) on the membrane, search for >500 μm suspected microplastics in the field of view, and pick them onto another new mixed cellulose filter membrane, making annotations. Select various typical characteristic suspected microplastics on the new membrane, transfer them by sampling needle to the ATR crystal surface, select the wavenumber range from 4000 to 400 cm⁻¹, and collect the spectrum. A similarity>70% in the spectral library is considered as microplastic. Then return to the spectrogram image interface, set the infrared imaging parameters of the FTIR microscope, and select the imaging area of corresponding size to obtain the infrared imaging component distribution map of the whole microplastics.

(12) Laser infrared imaging and component spectrum of microplastics on the first-size purified filter membrane (10-500 μm): Place the re-extracted first-size purified filter membrane obtained from step (11) into a 50 mL beaker, use filtered anhydrous ethanol solution to immerse the membrane surface, oscillate on a shaker at 100 rpm for 12 h, rinse both sides and gap edges of the membrane multiple times, concentrate the extraction solution to a 100 μL suspension at 60° C. on a constant-temperature heating plate, add the suspension dropwise onto a clean surface of the high-reflective glass, transfer the high-reflective glass into the sample chamber of the laser infrared spectrometer, set spectral parameters, capture the infrared spectrum of all 10-500 μm microplastics on the membrane surface, and set the spectral library match rate>0.7 to obtain the infrared imaging maps and polymer types of all microplastics on the membrane.

(13) Small-size microplastic characteristic observation, Raman imaging, and component spectrum (1-10 μm): Place the second-size purified filter membrane obtained from step (7) on the stage of the Raman spectrometer, select 785 nm as the excitation wavelength, set the scanning range at 500-3000 cm⁻¹, set the integration time at 5 s, set the integration number at 3, scan the membrane surface in a “Z” pattern, and identify all 1-10 μm suspected microplastics on the membrane, with spectrum library match rate>70% considered as microplastic. Record the type, color, morphology, size, and other morphological characteristics of each microplastic. Use the Raman Mapping accessory function to define the surface scan area, perform pathway Raman scanning across the whole membrane surface to create a stitched Raman imaging distribution map approximating the membrane surface.

(14) Submicron-size microplastic characteristic observation, Raman microscopic imaging, and component spectrum (0.1-1 μm): Place the third-size purified filter membrane obtained from step (7) on the stage of the Raman imaging microscope, calibrate the membrane background, set the spectral range at 500-4000 cm⁻¹, set the integration time at 1 s, select the surface scan mode, define the surface scan area, perform Raman microscopic identification of 0.1-1 μm suspected microplastics on the whole membrane surface, and automatically stitch into the complete Raman microscopic imaging map approximating the membrane surface. Set the database match rate>70% as microplastic, search for microplastics on membrane, and record the size, color, type, and shape information of each microplastic.

(15) Quality control protocol: This quality control protocol intends to use a standard addition recovery experiment to verify the accuracy of this method. The experimental group first carries out steps (1)-(3) of hierarchical extraction to obtain each freeze-dried mussel tissue after experimental pretreatment. The control group starts from step (4) above, and all subsequent treatment operations are the same as in the experimental group. The experimental standards use standard yellow-green fluorescent PE microspheres of 20±10 μm, 50±20 μm, 100±20 μm, 200±50 μm, and 500±50 μm, mixed and configured according to the following compositional formula for microplastic types adsorbed to deep-sea extreme environment bivalves. The self-formulated size distribution formula of microplastics in bivalves in the present embodiment is shown in Table 1 below:

TABLE 1
Size distribution formula of microplastics in bivalves

| | 20 ± 10 μm | 50 ± 20 μm | 100 ± 20 μm | 200 ± 50 μm | 500 ± 50 μm | |
|---|---|---|---|---|---|---|
| C, % | 28 | 41 | 24 | 5.4 | 1.6 | 100 |

The yellow-green fluorescent PE microsphere standards of 20±10 μm, 50±20 μm, 100±20 μm, 200±50 μm, and 500±50 μm are sequentially pre-screened through 800 mesh, 300 mesh, 150 mesh, 70 mesh, and 32 mesh stainless steel sieves to remove microplastic particles with negative particle size deviations from these five size categories. Then, using an analytical balance with one-millionth precision, proportionally weigh 280 μg of 20-30 μm, 410 μg of 50-70 μm, 240 μg of 100-120 μm, 54 μg of 200-250 μm, and 16 μg of 500-550 μm screened yellow-green fluorescent PE microspheres, and mix the five sizes of microspheres uniformly in a clean beaker to prepare 1 mg of mixed fluorescent microplastic powder.

According to the dry weight of each biological tissue, place each biological tissue in a clean 50 mL or 100 mL ground-glass stoppered conical flask, add a certain amount of trypsin solution for digestion at a ratio of 1 g tissue dry weight to 30 mL enzyme digestion solution, and add the microplastic powder proportionally to each tissue enzyme digestion solution at a ratio of 1 g tissue dry weight to 1 mg mixed fluorescent microplastic powder. After sealing the system, digest at 37° C. on a constant temperature shaker at 100 rpm for 24 h. The subsequent pH-enhanced digestion, filtration, extraction, and identification steps are the same as the above steps. During the identification step using microscopic observation, it is necessary to specifically select plastic microspheres that emit yellow-green fluorescence, while other suspicious microplastics are not included in the identification system. The objective of this quality control protocol is to compare the characteristic microplastic particles identified by digestion using the present application with the known sample addition amounts, highlighting the feasibility of this method for non-destructive size-based extraction of microplastics from biological tissues in extreme ecosystems. The experimental results are shown in Table 2 below:

TABLE 2
Spiked recovery results of mixed fluorescent microplastic powder

| | | | 20-30 | 50-70 | 100-120 | 200-250 | 500-550 | | |
|---|---|---|---|---|---|---|---|---|---|
| | Tissue | | μm PE | μm PE | μm PE | μm PE | μm PE | Final | |
| Dissected | dry | Powder | recovery | recovery | recovery | recovery | recovery | recovery | Recovery |
| tissue | weight/g | dosage/mg | mass/μg | mass/μg | mass/μg | mass/μg | mass/μg | mass/mg | rate/% |
| Gills | 1.6189 | 1.619 | 412 | 614 | 370 | 72 | 21 | 1.489 | 91.99% |
| Mantle | 3.4654 | 3.465 | 948 | 1251 | 792 | 159 | 45 | 3.195 | 92.21% |
| Lips | 0.0863 | 0.086 | 20 | 31 | 18 | 4 | 1 | 0.074 | 85.66% |
| Foot | 0.2928 | 0.293 | 79 | 106 | 64 | 12 | 4 | 0.265 | 90.44% |
| Intestines | 0.0222 | 0.022 | 6 | 8 | 4 | 1 | 0 | 0.019 | 85.63% |
| Visceral | 0.9515 | 0.952 | 248 | 365 | 206 | 38 | 11 | 0.870 | 91.38% |
| mass | | | | | | | | | |
| Adductor | 0.6785 | 0.679 | 179 | 254 | 142 | 27 | 9 | 0.611 | 89.98% |
| muscle | | | | | | | | | |

### Comparative Embodiment 1

Same as Embodiment 1, with the difference being no enzyme solution digestion, instead using an acid/alkali digestion method.

### Comparative Embodiment 2

Same as Embodiment 1, with the difference being no pH phased enhancement.

### Comparative Embodiment 3

Same as Embodiment 1, with the difference being no hydrogen peroxide digestion.

Comparison of Comparative Embodiments 1-3 and Embodiment 1: \textcircled{1} No enzyme solution digestion was used. The method in Comparative Example 1 would cause chemical degradation of microplastic types such as PC, PET, and PA in bivalve tissues, with surface morphology being eroded, resulting in false positive results for in-situ degraded microplastics in deep-sea bivalves. \textcircled{2} No pH adjustment solution was used to enhance enzymatic reactions. Deep-sea bivalve cells have evolved tough protein support structures, and thermostable proteins with stronger resistance to denaturation and degradation can weaken the decomposition effect of proteases, necessitating enhancement measures different from those used offshore. \textcircled{3} No hydrogen peroxide solution was used to digest remaining tissue condensates. During the enhanced protease separation process of bivalve tissues, some tough protein complexes still remain, and only more intense oxidation reactions can completely remove the remaining complex organic matter to expose the contained microplastics without damage and ensure purity. These three digestion steps complement each other and are indispensable, synergistically improving the digestion efficiency of organic matter in bivalve tissues. Trypsin initially breaks down protein barriers, pH-enhanced enzyme solution further decomposes complex organic matter, and finally hydrogen peroxide thoroughly oxidizes remaining stubborn organic matter. This multi-step combined progressive digestion method can effectively remove resistant organic matter from tissue cells, allowing full-scale microplastics to be extracted sequentially in a nearly undamaged state, fulfilling the mission of size-based identification.

Additionally, the prior art commonly uses mechanical crushers to homogenize all tissues, causing microplastics adsorbed or degraded within tissues to be cut into particles with uniform texture and smaller particle sizes. Moreover, most techniques use single identification methods to obtain microplastic information within fixed size ranges. Compared to the prior art, Embodiment 1 does not require a crusher to expand the enzymatic reaction area of bivalve tissues through cutting, as the digestion efficiency using the three overlapping digestion methods is sufficient to achieve the same effect. Combined with the proposed advantageous instrument combination identification protocol, identification of full-scale microplastics can be achieved, broadening the detection range of previous technologies.

### Embodiment 2

Same as Embodiment 1, with the differences being:

Enzyme solution pre-digestion: Add trypsin solution for digestion to each biological tissue at the ratio of 1 g dry weight to 30 mL trypsin solution, and digest at 35° C. for 30 h.

pH phased enhancement: Use pH adjustment solution to adjust the pH to 7.5, then add trypsin solution multiple times into the enzyme digestion solution to accelerate filamentous condensate disintegration to obtain an secondary enzyme digestion solution, each added trypsin solution volume being ⅓ of the enzyme solution volume in step S2.

30% hydrogen peroxide digestion: Pick out filamentous condensates, add 30% hydrogen peroxide solution, and digest residual cell adhesives at 65° C. for 2 h.

### Embodiment 3

Same as Embodiment 1, with the differences being:

Enzyme solution pre-digestion: Add trypsin solution for digestion to each biological tissue at the ratio of 1 g dry weight to 40 mL trypsin solution, and digest at 40° C. for 24 h.

pH phased enhancement: Use pH adjustment solution to adjust pH to 7.5, then add trypsin solution multiple times into the enzyme digestion solution to accelerate filamentous condensate disintegration to obtain secondary enzyme digestion solution, each added trypsin solution volume being ¼ of the enzyme solution volume in step S2.

30% hydrogen peroxide digestion: Pick out the filamentous condensates, add 30% hydrogen peroxide solution, and digest residual cell adhesives at 60° C. for 4 h.

The descriptions of the above embodiments are only to help understand the technical protocol of the present application and its core ideas. It should be pointed out that for technicians of ordinary skill in the art, on the premise of not departing from the principles of the present application, several improvements and modifications can also be made, which also fall within the protection scope of the claims of the present application.

What is claimed is:

1-10. (canceled)

11. A size-based hierarchical extraction and identification method for microplastics in bivalves from deep-sea methane seeps, comprising the following steps:
S1, frozen preservation: freezing retrieved bivalves for preservation;
S2, tissue pretreatment: after the bivalves frozen for preservation in step S1 undergo constant temperature thawing and closed dissection, obtaining various biological tissues of the bivalves, and freeze-drying the various biological tissues of the bivalves for later use;
S3, enzyme solution pre-digestion: adding trypsin solution for digestion to the various biological tissues of the bivalves obtained in step S2 to perform digestion, to obtain a primary enzyme digestion solution with numerous tissue filamentous condensates;
S4, pH phased enhancement: using pH adjustment solution to adjust a pH of the primary enzyme digestion solution obtained in step S3 to 7.5, then adding trypsin solution to the enzyme digestion solution multiple times to accelerate a breakdown of filamentous condensates, to obtain a secondary enzyme digestion solution, a volume of trypsin solution added each time being ⅓ to ¼ of a volume of the trypsin solution for digestion in step S3; wherein the pH adjustment solution in step S4 is potassium dihydrogen phosphate solution with a mass concentration of 0.0136 g/mL or potassium hydroxide solution with a mass concentration of 0.0562 g/mL; the trypsin solution is prepared by the following steps: weighing 6.80 g potassium dihydrogen phosphate, adding 500 mL water to dissolve it, adjusting pH to 7.5 with 0.1 mol/L potassium hydroxide solution, adding 30.00 g trypsin, dissolving with water, then diluting to 1 L to obtain the trypsin solution;
S5, 30% hydrogen peroxide digestion: picking out the filamentous condensates from the secondary enzyme digestion solution obtained in step S4, adding 30% hydrogen peroxide solution by mass percentage to the filamentous condensates, and digesting remaining cellular adhesive materials at 60-65° C. for 2-4 hours until no bubbles are generated in a digestion solution, wherein complete digestion is achieved to obtain a nearly transparent solution with no obvious suspended matter, to obtain a hydrogen peroxide digestion solution;
S6, mixed digestion solution size-based extraction: mixing the secondary enzyme digestion solution obtained in step S4 and the hydrogen peroxide digestion solution obtained in step S5, performing first-size extraction to enrich all target objects>10 μm onto a filter membrane, to obtain a first-size extraction filter membrane; then performing second-size extraction on a first-size filtrate to enrich all target objects of 1-10 μm onto a filter membrane, to obtain a second-size extraction filter membrane; then performing third-size extraction on a second-level filtrate to enrich all target objects of 0.1-1 μm onto a filter membrane, to obtain a third-size extraction filter membrane;
S7, subjecting microplastics on the first-size extraction filter membrane, the second-size extraction filter membrane, and the third-size extraction filter membrane obtained in step S6 to characteristic observation, microscopic imaging, and compositional spectral detection in sequence to obtain size-based abundance information of full-scale microplastics in the various biological tissues in the bivalves, wherein specific steps are as follows:
S71, separating the first-size extraction filter membrane into a large-size microplastic filter membrane with particle size greater than 500 μm and a medium-size filter membrane with particle size of 10-500 μm; performing infrared imaging on microplastics in the large-size microplastic filter membrane with particle size greater than 500 μm and qualitatively analyzing their distribution to obtain a compositional type uniformity of all suspected microplastics and determine types of these polymers; performing microscopic infrared imaging on microplastics in the medium-size filter membrane with particle size of 10-500 μm and qualitatively analyzing their composition, and calculating their technical parameters comprising particle count, particle diameter, and equivalent area;
S72, performing morphological observation, microscopic imaging, and compositional analysis on small-size microplastics with particle size of 1-10 μm in the second-size extraction filter membrane, counting size, color, shape, equivalent diameter, and particle count of all small-size microplastics, and performing Raman imaging to obtain compositional distribution information;
S73, performing morphological observation, microscopic imaging, and compositional analysis on submicron-size microplastics with particle size of 0.1-1 μm in the third-size extraction filter membrane, counting size, color, shape, and particle count of all submicron-size microplastics, and performing microscopic imaging to obtain compositional distribution information.

12. The method according to claim 11, wherein specific steps of step S1 are: selecting undamaged bivalves, washing the bivalves multiple times until adsorbed mud on a surface is removed, then placing cleaned bivalves into sterile containers and preserving frozen at −20° C.

13. The method according to claim 11, wherein specific steps of step S2 are: thawing the bivalves frozen and stored in step S1 at a constant temperature of 4° C. for 0.5-1.5 h, then after closed dissection, obtaining the various biological tissues of the bivalves; washing the various biological tissues of the bivalves with a washing solution to remove seawater microplastics and various impurities attached outside bivalve tissue cells, then freezing and preserving them at −80° C. for 5-7 h, and freeze-drying at −60° C. for 20-28 h for later use.

14. The method according to claim 13, wherein the washing solution is prepared by the following steps: adding 5.04 g sodium chloride powder, 0.06 g barium chloride powder, 0.12 g ferrous sulfate powder, 0.02 g manganese sulfate powder, 1.2 g magnesium chloride powder, 0.4 g potassium chloride powder, 0.4 g calcium chloride powder, 2.6 mg sodium nitrate powder, 22 mg sodium silicate powder, 1 mg sodium dihydrogen phosphate powder, and 2.5 g sodium sulfate powder to 800 mL of ultrapure water, dropwise adding 23.42 mL of concentrated hydrochloric acid while continuously shaking and stirring, then diluting to 1 L and filtering several times using 0.1 μm aqueous microporous filter membrane to obtain the washing solution.

15. The method according to claim 11, wherein specific steps of step S3 are as follows: adding the trypsin solution for digestion to the various biological tissues of the bivalves obtained in step S2 at a ratio of 1 g of dry weight of biological tissue to 30-40 mL of trypsin solution, and performing digestion at 35° C.-40° C. for 24-30 hours to obtain the primary enzyme digestion solution with numerous tissue filamentous condensates.

16. The method according to claim 11, wherein in step S6, the first-size extraction uses a glass filter core with diameter of 50 mm paired with a stainless steel membrane with diameter of 47 mm and mesh size of 1200; the second-size extraction uses a filter core with diameter of 25 mm paired with a glass microfiber filter membrane with diameter of 25 mm and pore size of 1 μm; the third-size extraction uses a filter core with diameter of 25 mm paired with an inorganic aluminum oxide membrane with diameter of 25 mm and pore size of 0.1 μm.

17. The method according to claim 11, wherein step S7 further comprises: S74, extracting the microplastics of each size separately, and using ATR-FTIR, LDIR, Raman, and micro-Raman spectrometers; wherein the total measured microplastics represent a full-scale microplastic content of a specific tissue in a single bivalve; combined with abundance correction in the experimental steps, obtaining the size-based abundance information of full-scale microplastics in the various biological tissues in the bivalves.
